(19) United States
(12) Patent Application Publication
(10) Pub. No.: US 2025/0369924 A1
(43) Pub. Date: Dec. 4, 2025

(54) LASER ACOUSTIC RESONANCE SPECTROSCOPY BASED NON-DESTRUCTIVE DIAGNOSTIC TECHNIQUES

(71) Applicant: METROLASER, INC., Laguna Hills, CA (US)

(72) Inventor: Brandon Leu, Aliso Viejo, CA (US)

(21) Appl. No.: 19/181,197

(22) Filed: Apr. 16, 2025

Related U.S. Application Data
(60) Provisional application No. 63/654,858, filed on May 31, 2024.

(51) Int. Cl. G01N 29/12; G01N 29/24; G01N 29/46; G01N 33/00

(52) U.S. Cl. CPC G01N 29/12; G01N 29/2418; G01N 29/46; G01N 33/0078; G01N 2291/014; G01N 2291/0289

(57) ABSTRACT

A system and a method for non-destructive characterizations of objects using Laser Acoustic Resonance Spectroscopy (LARS)-based diagnostic techniques are provided. The system includes using a laser doppler vibrometer to measure vibrational responses of objects. The method includes measuring vibrational frequency responses of a first object and a second object, performing a spectra analysis of the vibrational frequency responses, determining a frequency shift based on the spectra analysis, and indicating a difference between the first object and the second object or a presence of a defect in the second object if the determined frequency shift exceeds a predefined threshold value. The difference between the first object and the second object may indicate the presence of a void, a crack, or a plurality of pores in the second object, or can be used for validating or authenticating the second object as a defective or counterfeit item, in various embodiments.

### CROSS REFERENCE TO RELATED APPLICATION

This application claims priority to U.S. Patent Application No. 63/654,858, filed May 31, 2024, all of which is incorporated by reference herein in its entirety.

### GOVERNMENT SPONSORED RESEARCH

The present disclosure includes work commenced with the support from Missile Defense Agency Phase II SBIR Contract No. HQ0860-22-C-7124, “Non-Destructive Evaluation of Additive Manufacturing Parts Using Resonance Spectroscopy”. The government has certain rights in the invention.

### FIELD OF INVENTION

Embodiments of the present disclosure relate generally to Laser Acoustic Resonance Spectroscopy (LARS)-based diagnostic techniques, and more particularly, for example, to a system and a method for non-destructive characterizations of objects using LARS.

### BACKGROUND

Reproducibility of parts is the hallmark of the industrial age. In a typical production of metal or alloy parts, a sufficient quality control may be needed in the manufacturing process with high reproducibility, which may involve systematic procedures aimed at verifying and maintaining a desired level of quality in the manufactured products. This encompasses the entirety of the manufacturing process, from the initial design to the final product inspection. The ability to measure parts and provide assurance that they have been produced according to their specifications is another crucial aspect of manufacturing quality control.

Although a plethora of measurements can be used to investigate a manufactured metal alloy part, there are only a few non-destructive characterization techniques that are suitable for rapid investigations, for example, of additively manufactured parts with unconventional geometric shapes. Nonetheless, with the advances and prolificity of additive manufacturing tools (or generically referred to as 3D printers), the requisite for quality control of the additively manufactured parts also rises to ensure that such 3D printed parts are of the same or substantially similar quality, and free of defects. Thus, there is a need for a system and/or a method that can help with non-destructive QC processes to ensure a high reproducibility in metal alloy additive manufacturing.

### SUMMARY

In accordance with one or more embodiments, a method is provided. The method may be used for non-destructive characterizations of objects using diagnostic techniques as disclosed herein. The method may include measuring a first vibrational frequency response of a first object produced via a first process; measuring a second vibrational frequency response of a second object produced via a second process; performing a spectra analysis of the first vibrational frequency response and the second vibrational frequency response; determining a frequency shift based on the spectra analysis; and/or indicating a presence of a defect in the second object if the determined frequency shift exceeds a predefined threshold value.

In various embodiments, performing the spectra analysis of the first vibrational frequency response and the second vibrational frequency response may include identifying a first plurality of peaks from the first vibrational frequency response as resonant frequency modes of the first object; identifying a second plurality of peaks from the second vibrational frequency response as resonant frequency modes of the second object; for each resonant frequency mode of the first (second) object, comparing a peak position of a corresponding peak of the first plurality and a peak position of a corresponding peak of the second plurality; for each resonant frequency mode of the first (second) object, determining a frequency difference between the two peak positions; and averaging all determined frequency differences to provide the frequency shift for the first vibrational frequency response and the second vibrational frequency response.

In one or more embodiments, the first object and the second object may have a substantially similar geometrical shape or a substantially similar volume. In one or more embodiments, the first object and the second object may have a substantially similar geometrical shape, a substantially similar volume, and a substantially similar material composition. In one or more embodiments, the first object and the second object may have the same metals. In one or more embodiments, the first object and the second object may have different heat treatment profiles or processing history. In one or more embodiments, the defect in the second object may include a void, a crack, or a plurality of pores. In one or more embodiments, the presence of the defect in the second object may indicate that the defect is a physical defect in the second object and that the physical defect is not present in the first object.

In one or more embodiments, the first process is a metallurgical process, and the second process is an additive manufacturing process. In one or more embodiments, the first process and the second process are both metallurgical processes. In one or more embodiments, the first process is a first additive manufacturing process using a laser-based three-dimensional (3D) printer with a first set of manufacturing parameters, the second process is a second additive manufacturing process using the laser-based 3D printer with a second set of manufacturing parameters, and the first set of manufacturing parameters may differ from the second set of manufacturing parameters in laser power, laser raster speed, laser spot size, or a combination thereof. In one or more embodiments, the presence of the defect in the second object may indicate a difference in porosity or material composition between the first object and the second object.

In various embodiments, the method may further include validating the second object as a defective item based on the presence of the defect in the second object. In various embodiments, the method may further include authenticating the second object as a counterfeit item based on the presence of the defect in the second object.

In one or more embodiments, the first vibrational frequency response of the first object and the second vibrational frequency response of the second object are measured via a laser acoustic resonance spectroscopy system using a laser doppler vibrometer. In one or more embodiments, the first vibrational frequency response of the first object and the second vibrational frequency response of the second object are measured in an acoustic and ultrasonic frequency range between 1 Hz and 60 kHz.

In accordance with one or more embodiments, a system is provided. The system may be configured for non-destructive characterizations of objects using diagnostic techniques as disclosed herein. The system may include a laser doppler vibrometer configured to measure a vibrational response of a first object and a second object; a processor and a non-transitory computer readable medium operably coupled thereto, the processor operationally coupled and configured to control the laser doppler vibrometer and to acquire data from the laser doppler vibrometer, wherein the non-transitory computer readable medium comprising a plurality of instructions stored in association therewith that are accessible to, and executable by, the processor, to perform one or more operations. Such operations may include acquiring a first vibrational frequency response of the first object; acquiring a second vibrational frequency response of the second object; performing a spectra analysis of the first vibrational frequency response and the second vibrational frequency response; determining a frequency shift based on the spectra analysis; and/or indicating a presence of a defect in the second object if the determined frequency shift exceeds a predefined threshold value.

In one or more embodiments, performing the spectra analysis of the first vibrational frequency response and the second vibrational frequency response may include identifying a first plurality of peaks from the first vibrational frequency response as resonant frequency modes of the first object; identifying a second plurality of peaks from the second vibrational frequency response as resonant frequency modes of the second object; for each resonant frequency mode of the first (second) object, comparing a peak position of a corresponding peak of the first plurality and a peak position of a corresponding peak of the second plurality; for each resonant frequency mode of the first (second) object, determining a frequency difference between the two peak positions; and averaging all determined frequency differences to provide the frequency shift for the first vibrational frequency response and the second vibrational frequency response.

In various embodiments, the system may further include an elastic mesh net configured to mount the first object or the second object; a piezoelectric transducer disposed underneath the elastic mesh net and configured to vibrate the first object and/or the second object during a vibrational response measurement performed by the laser doppler vibrometer. In one or more embodiments, the laser doppler vibrometer may be further configured to measure the vibrational response in an acoustic frequency range between 1 Hz and 60 kHz.

In one or more embodiments, the presence of the defect in the second object may indicate a void, a crack, or a plurality of pores, a difference in porosity or material composition between the first object and the second object, that the defect is a physical defect in the second object and that the physical defect is not present in the first object, or a combination thereof. In one or more embodiments, the presence of the defect in the second object may indicate that the second object is a defective item or a counterfeit item.

In accordance with one or more embodiments, a method is provided. The method may be used for non-destructive characterizations of objects using diagnostic techniques as disclosed herein. The method may include measuring vibrational frequency responses of a first object and a second object; performing a spectra analysis of the vibrational frequency responses; determining a frequency shift based on the spectra analysis; and/or indicating a difference between the first object and the second object if the determined frequency shift exceeds a predefined threshold value.

In various embodiments, performing the spectra analysis of the vibrational frequency responses may further include identifying a plurality of peaks from the vibrational frequency responses; pairing corresponding peaks originating from the first object and the second object; designating the paired corresponding peaks as resonant frequency modes of the first object and second object; for each resonant frequency mode of the first object and the second object, determining a frequency difference for each of the paired corresponding peaks; and averaging all determined frequency differences to provide the frequency shift.

In one or more embodiments, the first object and the second object may have a substantially similar geometrical shape or a substantially similar volume. In one or more embodiments, the first object and the second object may have a substantially similar geometrical shape, a substantially similar volume, and a substantially similar material composition. In one or more embodiments, the first object and the second object may have the same metals. In one or more embodiments, the first object and the second object may have different heat treatment profiles or processing history.

In one or more embodiments, the difference between the first object and the second object may indicate a presence of a void, a crack, or a plurality of pores in the second object. In one or more embodiments, the difference between the first object and the second object may indicate a physical defect in the second object and that the physical defect is not present in the first object. In one or more embodiments, the first object may be produced via a metallurgical process, and the second object may be produced via an additive manufacturing process.

In one or more embodiments, the first object may be produced via a laser-based three-dimensional (3D) printer with a first set of manufacturing parameters, the second object may be produced via the laser-based 3D printer with a second set of manufacturing parameters, and the first set of manufacturing parameters may differ from the second set of manufacturing parameters in laser power, laser raster speed, laser spot size, or a combination thereof. In one or more embodiments, the difference between the first object and the second object may indicate a difference in porosity or material composition between the first object and the second object.

In various embodiments, the method may further include validating the second object as a defective item based on the indicated difference between the first object and the second object. In various embodiments, the method may further include authenticating the second object as a counterfeit item based on the indicated difference between the first object and the second object.

In one or more embodiments, the vibrational frequency responses of the first object and the second object may be measured via a laser acoustic resonance spectroscopy system using a laser doppler vibrometer. In one or more embodiments, the vibrational frequency responses of the first object and the second object may be measured in an acoustic frequency range between 1Hz and 60 kHz.

### BRIEF DESCRIPTION OF THE DRAWINGS

For a more complete understanding of the principles disclosed herein, and the advantages thereof, reference is now made to the following descriptions taken in conjunction with the accompanying drawings, in which:

FIG. 1 illustrates a schematic of a system for performing non-destructive investigations of an object, in accordance with various embodiments.

FIG. 2 shows a plot of a resonant frequency response of an object measured using LARS technique, in accordance with various embodiments.

FIG. 3 shows a plot depicting a comparison of a resonant spectrum from a first object (e.g., a reference part) and a resonant spectrum from a second object (e.g., an identical part with a known internal defect), in accordance with various embodiments.

FIG. 4 shows another plot depicting a zoomed-in section of the plot shown in FIG. 3.

FIG. 5A depicts an array of additively manufactured dogbone samples, in accordance with various embodiments.

FIG. 5B depicts a shape of an additively manufactured dogbone sample, in accordance with various embodiments.

FIG. 6 depicts a plurality of X-ray computed tomography (CT) scans showing porosity evolution in samples created with different relative laser power using laser powder bed fusion, in accordance with various embodiments.

FIG. 7 shows a plot depicting pore volume for all the groups of dogbone samples 500, which have been measured and plotted together to illustrate the porosity evolution in the samples as functions of the printing parameters, in accordance with various embodiments.

FIG. 8 shows a plot depicting for the average SS for the heat treated and as-printed groups of dogbone samples based on laser power variation relative to the laser power used to manufacture the control dogbones, in accordance with various embodiments.

FIG. 9 shows a plot depicting for the average SS for the heat treated and as-printed groups of dogbone samples based on laser speed variation relative to the laser speed used to manufacture the control dogbones, in accordance with various embodiments.

FIG. 10 shows a plot depicting for the average SS for the heat treated and as-printed groups of dogbone samples based on laser spotsize variation relative to the laser spotsize used to manufacture the control dogbones, in accordance with various embodiments.

FIG. 11A shows a plot depicting partial LARS measured resonant frequency spectra of three control group dogbones overlayed together, in accordance with various embodiments.

FIG. 11B shows a laser powder bed fusion (LPBF) print layout of the printed dogbones, in accordance with various embodiments.

FIG. 12 is a block diagram illustrating an example computer system with which embodiments of the disclosed system and method may be implemented, in accordance with various embodiments.

FIG. 13 illustrates a flowchart for a method, in accordance with one or more embodiments.

FIG. 14 illustrates a flowchart for another method, in accordance with one or more embodiments.

It is to be understood that the figures are not necessarily drawn to scale, nor are the objects in the figures necessarily drawn to scale in relationship to one another. The figures are depictions that are intended to bring clarity and understanding to various embodiments of apparatuses, systems, and methods disclosed herein. Wherever possible, the same reference numbers will be used throughout the drawings to refer to the same or like parts. Moreover, it should be appreciated that the drawings are not intended to limit the scope of the present teachings in any way.

### DETAILED DESCRIPTION

In accordance with various embodiments, a system and a method for non-destructive investigations of objects are described. The disclosed system and method can be used for non-destructive quality control (QC) processes to ensure a high reproducibility in metal or alloy parts, such as those produced via additive manufacturing, such as, for example, three-dimensional (3D) printing tools. As disclosed herein, the system and method may include using Laser Acoustic Resonance Spectroscopy (LARS)-based diagnostic technique to facilitate material analyses and characterizations. In one or more disclosed embodiments, the LARS-based diagnostic technique (also interchangeably referred to herein as LARS technique) may be used as a novel non-destructive investigative technique for detection of internal defects and imperfections, namely, voids, cracks, and pores, in metal objects or parts that are produced, for example, via an additive manufacturing (AM), or 3D printing, tool. Although the AM-produced objects or parts may appear pristine on the surface, they may contain internal voids, pores, or cracks. These internal imperfections, voids, pores, or cracks within the AM-produced objects/parts (also interchangeably referred to herein as AM objects/parts) may be detected via the LARS technique, as disclosed herein. In addition, in some embodiments, the LARS technique may be used to guide the optimization of the AM objects/parts by detecting embedded or hidden imperfections and defects by optimizing printing parameters for the AM tool to minimize the defects/imperfections within the printed objects/parts. In some embodiments, the LARS technique may also be used for rapid screening of the AM objects/parts and identifying those parts with greater amounts of defects that could potentially lead to premature failures. In some embodiments, the disclosed LARS technique may be particularly useful for QC processes of mass-produced AM objects/parts that require rapid and non-destructive evaluations before they are put into service.

In some embodiments, the disclosed system may include using a laser doppler vibrometer to measure vibrational responses of objects. The disclosed method may include, among many others, measuring vibrational frequency responses of objects, for example, a first object and a second object. Once the vibrational frequency responses are measured, the next step in the method is performing a spectra analysis of the vibrational frequency responses to determine a frequency shift between the two vibrational frequency responses based on the spectra analysis. From the frequency shift determined via the spectra analysis, a difference between the first object and the second object may be inferred. In some cases, the presence of a defect in the second object may be inferred from the spectra analysis, if the first object is used as a standard sample without any defects or imperfections. These determinations may be programmed via an algorithm in a computer to automatically provide the result using a predefined threshold value as a trigger when the determined frequency shift exceeds the threshold. As such, in various embodiments of the system and the method, the difference between the first object and the second object may indicate the presence of a defect, a void, a crack, or pores in the second object based on the analytical comparison between the first object and the second object. In some embodiments, the difference between the objects may be used for validating or authenticating the second object as a defective or counterfeit item, in various embodiments, if the first object is the standard sample, which is used, for example, as a pristine object without any defects.

In accordance with one or more embodiments, the LARS technique has been shown to reliably identify minor and major variations in porosity in parts that are near full density made by an AM technique called laser powder bed fusion (LPBF). In some embodiments, the porosity may originate from variations in the printing parameters used to make the parts, or from natural variations that occur over time during typical operating condition, such as inconsistencies in the powder bed surface, dirty lenses, fluctuations in laser power, laser printing speed, laser spot size, etc., or any other operating parameters in an AM tool.

Typically, the LARS technique works by comparing the resonance frequency of a suspect part against the baseline resonance frequency signature of a pristine part. The average frequency shift of all the resonance frequency modes between some frequency range can be directly used to predict the defect volume within the part, in accordance with one or more embodiments. The LARS technique may be used to operate in frequency ranges between about 1 Hz and about 60 kHz, which makes the LARS technique both an acoustic and ultrasonic technique. This technique can be used to identify the presence of internal voids or defects as small as 0.10 millimeters (mm) to about 0.6 mm in size, which may be suitable for detecting low void contents as small as 0.5% of the total volume of the part. The ability to detect such small internal voids and cracks with high confidence makes the LARS technique a unique and powerful tool for the non-destructive evaluation of additively manufactured materials. The LARS technique can be employed to quickly scan and identify parts that have larger defect content.

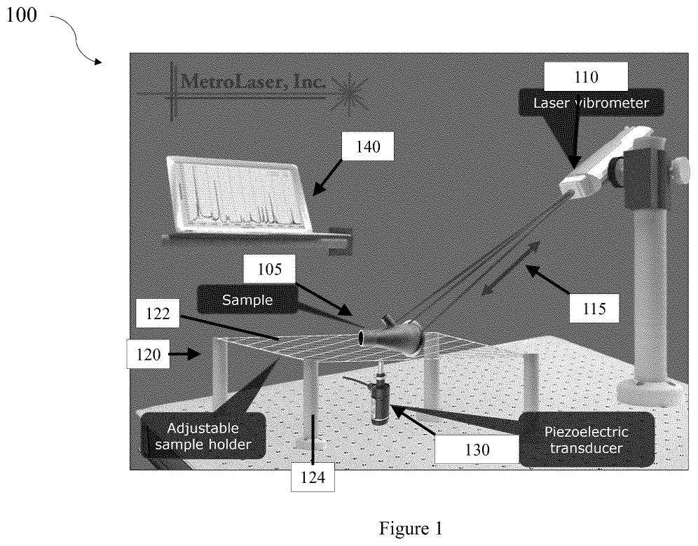

As discussed above, the LARS technique may be used in identifying anomalous or defective parts that have been made with additive manufacturing, in accordance with one or more embodiments. In one or more embodiments, the LARS technique can be especially powerful in identifying parts with a great porosity that have been made via laser powder bed fusion. In one or more embodiments, the LARS technique may be used to iteratively improve and guide the manufacturing process of parts made by laser powder bed fusion by rapidly and non-destructively characterizing changes in the porosity of samples made with varying printing parameters, thereby allowing for the faster and more efficient identification of optimal printing parameters that can be used to manufacture the part. The following detailed descriptions with respect to FIGS. 1-13 provide detailed information of the LARS technique.

FIG. 1 illustrates a schematic of a system 100 for performing non-destructive investigations of an object 105, in accordance with various embodiments. The system 100 may be configured for use in implementing non-destructive quality control (QC) processes to ensure a high reproducibility in metal or alloy parts produced via additive manufacturing or 3D printing tools, in accordance with one or more embodiments.

As illustrated in FIG. 1, the system 100 includes a laser doppler vibrometer 110, wherein the system 100 may be configured to perform a LARS-based diagnostic technique using the laser doppler vibrometer 110. In one or more embodiments, the system 100 may be configured to measure surface vibrations of the object 105, such as a funnel, using the laser doppler vibrometer 110 to obtain resonance frequency spectra 105 of the object 105. As further illustrated in FIG. 1, the system 100 includes a sample holder/stage assembly 120, which may include a net 122 with adjustable tension attached to a plurality of legs 124. In one or more embodiments, the object 105 may be mounted the tension-adjusted net 122 of the sample stage assembly 120 for LARS measurements. The system 100 also includes a transducer 130, such as a piezoelectric transducer or any other suitable transducer, to induce mechanical motion/vibration of the object 105, which can be detected via LARS measurements using the laser doppler vibrometer 110, in accordance with one or more embodiments.

As further illustrated in FIG. 1, the system 100 also includes a computing system, or simply, a computer 140, configured for processing of data acquired via LARS measurements using the laser doppler vibrometer 110, in accordance with one or more embodiments. The computer 140 may include a processor and a non-transitory computer readable medium operably coupled to the processor. In one or more embodiments, the processor/computer 140 may be configured to control the laser doppler vibrometer 110 and/or the transducer 130 to acquire data from the laser doppler vibrometer 110 for processing. In various embodiments, the system 100 include a plurality of instructions, which may be included on the non-transitory computer readable medium of the computer 140 to perform one or more operations pertaining to the LARS technique as described herein. The operational steps included in implementing the LARS measurement technique are described below in detail.

As illustrated in FIG. 1, the measurement set up includes the net 122, which may be an elastic mesh net that is stretched over the plurality of legs 124, typically four legs. The net 122 has sufficient elasticity such that when the object 105 is placed on the center of the net 122, the object 105 slightly sinks into the net and becomes cradled and stationary. The elasticity of the net 122 should not be so low that the object becomes engulfed by the net. The mounted object 105 may be stable and resistant to movements; both rigid body and rotational movements are minimized during LARS measurements, in one or more embodiments. The elasticity of the net 122 can be adjusted by tightening its connections to the legs 124. The net 122 is chosen as part of the sample holder/stage assembly 120 because it allows the target object 105 to vibrate freely with the least amount of influence from its boundary conditions, in one or more embodiments. Furthermore, the net 122 may allow for a general set up that can accommodate a wide range of object sizes and shapes with the least amount of adjustment to the sample holder/stage assembly 120.

In one or more embodiments, the transducer 130 may include a lead zirconate titanate (PZT) transducer. The transducer 130 may be used to induce mechanical vibration on the object 105 as described above with respect to FIG. 1. Once the target 105 has been mounted, the transducer 130 may be fixed below the object 105 in a position such that the transducer 130 makes contact with the bottom of the object 105 through a mesh opening in the net 122. In one or more embodiments, the object 105 may be firmly rest upon a tip of transducer 130 so that a direct contact is maintained during the measurement. The transducer 130 may vibrate the object 105 unidirectionally in a direction that is normal to the net 122, in one or more embodiments. In one or more embodiments, the transducer 130 is configured to stimulate the object 105 in a unidirectional, one bending mode may be excited, while the other bending, torsional, and/or extension modes may be ignored during the LARS measurements.

Upon mounting the object 105 to ensure a firm contact with the transducer 130 is established beneath the object 105, the transducer 130 can then be controlled electronically to induce a variety of different mechanical excitations, any of which may be defined by an operating conducting the LARS measurements. The various modes or variety of different mechanical excitations may include, for example, but not limited to a signal type, a trigger type, a frequency sweep range, a sweep time, and a voltage that is applied during the LARS measurements. A typical, and non-limiting, LARS measurement may be performed using the parameters for the transducer 130 as follows:

$$1 ) Signal type = sinusoidal wave ,$$

$$2 ) Trigger type = internal loop ,$$

$$3 ) Frequency range = 10 kHz - 60 kHz ,$$

$$4 ) Frequency sweep time = 50 milli seconds ,$$

$$and$$

$$5 ) Voltage = 10 V peak - to - peak .$$

An ideal setting for the transducer 130 may depend on which transducer is being used and the type of object being measured or inspected. Some transducers may have limited ranges of operable frequencies and voltages. In general, smaller objects resonate at higher frequencies and thus higher frequency ranges will be required as a result. Furthermore, smaller objects are most susceptible to rigid body motion if the stimulus from the transducer is too large, and therefore may require lower operating voltages. The opposite is true for larger objects, where larger objects resonate at lower frequencies and require more force to vibrate. Thus, larger objects tend to require lower frequency ranges and higher operating voltages of the transducer. It has been observed that lower frequency sweep times can offer higher signal-to-noise ratio. However, this may be adjusted based on the type of object being measured or inspected. It has been observed that 50 milli seconds is sufficient for most objects and even lower sweep times offer little improvement.

Once the object 105 is set up, e.g., mounted on the net 123 of the sample holder/stage assembly 120, LARS measurements may commence by activating the transducer 130 to induce vibrations. In one or more embodiments, the LARS measurements may be acquired from a single point on a surface of the object 105 using the laser doppler vibrometer 110, as illustrated in FIG. 1. The laser doppler vibrometer 110 is configured to operate by directing a laser beam on the surface of the vibrating object 105 and measuring the doppler shift of the scattered light caused by the vibration of the object 105. The measured frequency shifts are acquired via the processor/computer 140, and then used to calculate the velocity of the vibrating surface. The processor/computer 140 may be configured to produce, via a plurality of instructions, a correlation between the measured velocity and time, which may then be converted to a plot of velocity vs frequency to obtain the resonance frequency spectra of the object 105, in accordance with one or more embodiments.

In one or more embodiments, the LARS measurements are taken at multiple locations along the surface of the object 105 to ensure a high quality and representative vibrational spectrum is obtained. Depending on the size of the object 105, between 5-10 points along the sample may be selected for the measurements. In one or more embodiments, each individual measurement may be acquired using a low-pass filter whose value is set to the highest frequency value of the frequency sweep range. In some instances where the scattered light signals are low, the signals can be enhanced by coating the object 105 with white powder, tape, or chalk spray, if appropriate, in accordance with one or more embodiments described herein. Once the vibrational data at each point along the surface of the object 105 is measured and stored, the measured signals may be averaged together to obtain a single spectrum for each point and the peak locations are extracted for further analysis. The peak positions can be determined by setting a minimum relative amplitude threshold and minimum spacing between peaks.

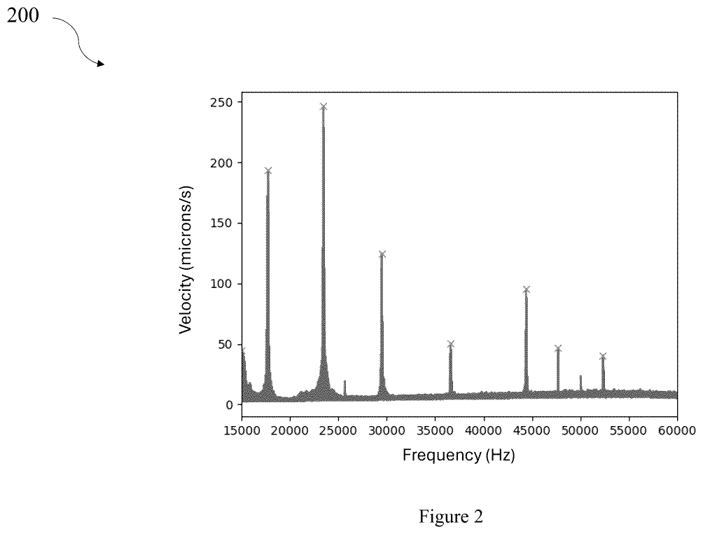

FIG. 2 shows a plot 200 of a resonant frequency response of an object measured using LARS technique, in accordance with various embodiments. The plot 200 depicts the resonant frequency response of the object, such as the object 105, showing a relationship between velocity values and frequency values for the object 105. As depicted in FIG. 2, seven resonant peaks, each of which are marked with a cross atop the peak, are identified in the resonant frequency response to correspond to seven resonant frequency modes of the object, such as the object 105. The resonant frequency response shown in the plot 200, for example, has been obtained by averaging 10 individual LARS measurements together, to improve the signal-to-noise ratio in the LARS measurements, in accordance with one or more embodiments.

The LARS measurements and analyses are performed as follows. In one or more embodiments, LARS technique may primarily operate as by pair-wise comparison of nearly identical parts. The technique may be useful for discerning differences in objects or parts that look alike or a have similar volume or a similar geometrical shape, but may have differences internally. LARS technique can be used to identify anomalous parts by comparing the resonance spectra of a suspicious part (e.g., a second object) against the baseline spectrum of a known pristine part (e.g., a first object), in one or more embodiments. The baseline spectrum can first be established by taking LARS measurements on an acceptable part (or the first object or the pristine object). The initial assessment of the acceptable part (or the first object or the pristine object) can be verified through other means, such as non-destructive investigation methods or otherwise, not mentioned here. Once the baseline spectrum has been acquired, it can be used to qualify other parts (e.g., a suspicious part or the second object) that are identical or substantially identical or similar in size, volume, and/or geometry. The resonant spectrum of a part/object is treated as a unique, identifying signature of the part/object that describes its size, shape, material, and condition. The resonant frequencies, ω, of a part scale according to Eq. 1, where k is the spring stiffness and m is the mass of the object.

$$ω \sim \sqrt{\frac{k}{m}} \qquad Eq . 1$$

According to Eq. 1, any changes in the mass or the stiffness of the object will result in changes in the natural frequency of the object. This forms the basis for the LARS analysis and allows the LARS technique to differentiate between, among many others:
1) Part/objects with even slight differences in geometry.
2) Part/objects of different sizes.
3) Part/objects that are nearly identical or identical in geometry, volume, or geometrical shape, but different in material composition.
4) Part/objects that are nearly identical or identical in geometry, volume, or geometrical shape, but have internal differences, such as voids, cracks, or inclusions.

Since the part/object geometry directly determines the resonant frequencies of the part/object, as described by Eq. 1, LARS technique can be especially useful to rapidly evaluate identical parts/objects that are mass produced using traditional manufacturing methods or additive manufacturing 3D printing techniques, where slight variances and irregularities from the manufacturing process are anticipated and unavoidable. The mass of the parts/objects may be variable due to porosity, suboptimal densification, geometrical differences, or impurities of the part. The stiffness of the parts/objects may be variable due to porosity, cracks, impurities, poor quality materials, or different processing histories during manufacturing. Any of these changes can cause a part/object to fail to meet quality standards. LARS technique can be used to identify and or second object/defective part.

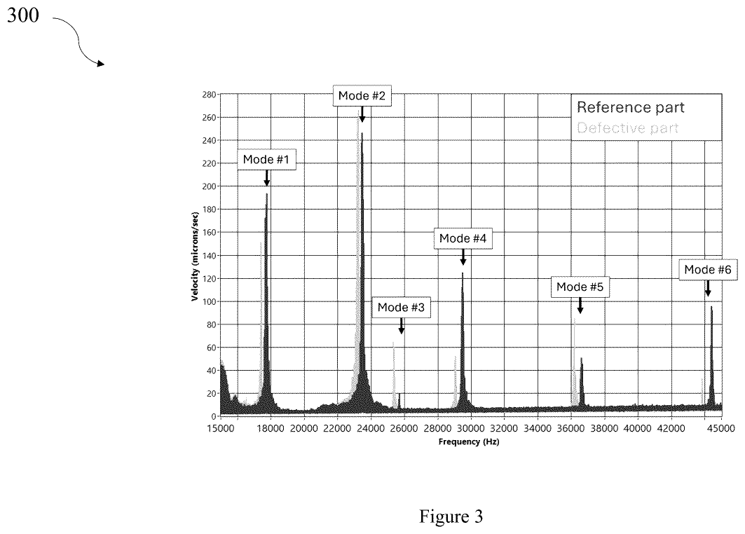

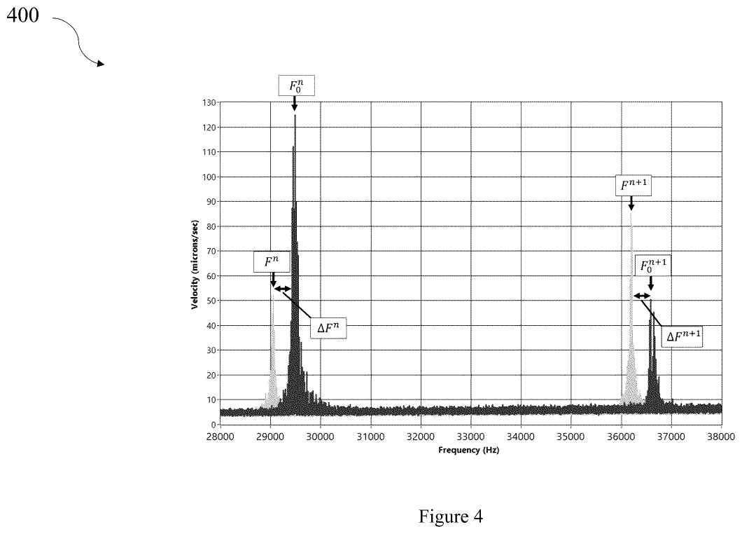

FIG. 4 shows a plot 400 depicting a zoomed-in section of plot 300 of FIG. 3. Specifically, the plot analysis begins by first obtaining a reference resonance spectrum from a well-qualified part/object that has acceptable levels of defects and degradations. The part/object can be treated as the standard for which other suspicious parts will be compared to. Tested parts/objects that are dissimilar to the standard part/object will have resonance spectra that are measurably different from the spectrum of the standard part/object and can be evaluated using LARS technique. Once the reference spectrum is obtained from a “good” part/object and the peak locations of the resonant frequency modes are recorded, then LARS technique can be used to rapidly qualify other “suspicious” parts/objects of the same geometry and material composition and categorize them as “good” or “bad”, depending on the needs and requirements of the user.

FIG. 3 shows a plot 300 depicting a comparison of a resonant spectrum from a first object (e.g., a reference part) and a resonant spectrum from a second object (e.g., a defective part, which is identical to the reference part, but with a known internal defect), in accordance with various embodiments. As shown in the plot 300 of FIG. 3, the resonant spectrum of the reference part (in red) and the resonant spectrum of the defective part (in green) include respective peaks for each of modes 1, 2, 3, 4, 5, and 6. Each peak in the spectra represents a different resonant frequency mode, which occurs at a specific frequency. As shown in FIG. 3, six of the resonant modes are detected in the frequency range between 15 kHz and 45 kHz. In one or more embodiments, a LARS measurement may contain between five and ten peaks to be used for analysis.

The plot 300 of FIG. 3 also depicts the differences in the resonant frequency for each of the identified modes 1-6. For example, the spectrum of the second object/defective part is downshifted from that of the first object/reference part. The down shift indicates that the peak positions of the second object/defective part occur at lower frequencies. The frequency shift, ΔFⁿ, for a particular frequency mode, n, can be calculated by simply taking the difference between respective peaks of the first object/reference part and second object/defective part, as shown below in Eq. 2.

$$Δ {F}_{i}^{n} = {F}_{i}^{n} - {F}_{0}^{n} \qquad Eq . 2$$

where i represents the number of suspicious or second objects/defective parts to be considered and n is the total number of resonant modes identified in both the first object/reference part and the second object/defective part. As disclosed herein,

$${F}_{0}^{n}$$

represents the frequency of n^{th} mode taken from the first object/reference part and

$${F}_{0}^{n}$$

is the frequency of the n^{th} mode taken from the i^{th} suspicious or second object/defective part.

FIG. 4 shows a plot 400 depicting a zoomed-in section of plot 300 of FIG. 3. Specifically, the plot 400 of FIG. 4 illustrates how a frequency shift is calculated for Mode #4and Mode #5 shown in the plot 300 of 3. In accordance with one or more embodiments, the frequency modes of the same mode number are used to calculate the frequency shift of a given mode. In other words, peak matching between the frequency modes of the first object/reference part and the suspicious or second object/defective part may be challenging, for example, if there are a lot of background noise during the measurement, which makes it hard to pinpoint the center of the peaks, if the signal quality between the reference and defective part are different from each other resulting in an unequal total number of identifiable peaks or different sets of resonant mode numbers in each part, and/or if there are many resonant peaks present in each part and they are closely spaced together. Thus, it is paramount for the LARS measurement technique to have high signal-to-noise ratio and be able to consistently measure all the resonant frequency modes of the object/part. The described sample holder/stage assembly 120 of FIG. 1 and measurement parameters disclosed above have been optimized to allow for reliable testing of a wide variety of objects/parts using the LARS technique.

As described herein, the frequency shift,

$$Δ {F}_{i}^{n} ,$$

can be airecuy used to quanry an object or a part. However, since LARS technique can operate on a wide range of frequencies, it is preferable to use relative frequency shifts for analysis, in accordance with one or more embodiments described herein. For example, the relative frequency shift, RFS, may be calculated by dividing the frequency shift by the frequency of the resonant mode of first object/reference part, as seen in Eq. 3. The RFSⁿ of each peak, n, can be expressed in terms of a percent shift from the first object/reference part.

$${RFS}^{n} = \frac{Δ {F}_{i}^{n}}{{F}_{0}^{n}} \times 100 \% \qquad Eq . 3$$

Lastly, the RFS of all the identified peaks in a resonance spectrum are averaged together to obtain a representative value of the spectral shift, SS, of a part, i, relative to the reference spectra of that part, see Eq. 4.

$${SS}_{i} = {\sum }_{1}^{n} {RFS}^{n} \qquad Eq . 4$$

For LARS analysis, the SS is used to determine the quality of a part. The magnitude of the SS is directly correlated to how different the inspected part (e.g., the second object, suspicious/defective part) is from the reference part (e.g., the first object). The sign of the SS, whether positive or negative, gives an indication of how the part may be different according to Eq. 1. A threshold value, SS^{threshold}, can be identified, beyond which, parts with SS greater than SS^{threshold} are deemed defective or out-of-tolerance, see Eq. 5. The exact value of the SS can be set to any value according to the needs and desired tolerances.

$$\vert {SS}_{i} \vert \geq {SS}^{threshold} \to Part i is defective ; \qquad Eq . 5$$

$$\vert {SS}_{i} \vert < {SS}^{threshold} \to Part i is not defective$$

To validate the LARS measurements and analyses thereof, various samples are prepared, tested, and analyzed in an experiment to demonstrate the capabilities of the LARS technique. The following investigation is performed to demonstrate and validate the ability of the LARS technique to detect defects, such as porosity, in additively manufactured AlSi₁₀Mg dogbone samples (simply interchangeably referred to herein as dogbones) made using laser powder bed fusion (LPBF) process. In order to create near-full-density objects/parts with desirable mechanical properties using the LPBF process, it is necessary to identify the optimal set of printing parameters for that specific part geometry and alloy composition. This is a time consuming and costly process that can be streamlined and optimized using the LARS (diagnostic) technique. The LARS-measured spectral shift, SS, is demonstrated to directly correlate with the pore volume in the printed dogbones, allowing for the rapid, non-destructive, and efficient identification of the optimal printing parameters used to manufacture objects/parts with the least porosity.

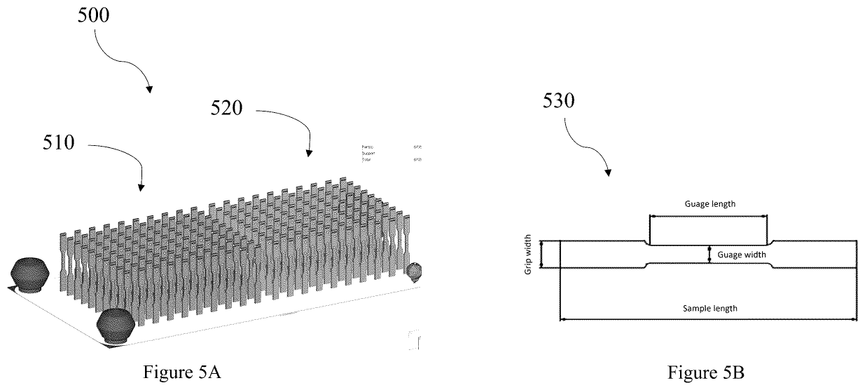

FIG. 5A depicts an array of additively manufactured dogbone samples 500, in accordance with various embodiments. FIG. 5B depicts a shape 530 of an additively manufactured dogbone sample, in accordance with various embodiments. As illustrated in FIG. 5A, the array of additively manufactured dogbone samples 500 includes 200 nearly identical dogbones, which are printed using the LPBF process and split into two identical sets 510 and 520 (which are shown in green and gray, respectively), with each set containing 100 dogbones. Within each set of 100, for example, in either set 510 or 520, the dogbones are further divided into several different groups, each made with a unique set of printing parameters. One set of 100 dogbones, for example set 510, may be heat treated at 300° C. for two hours while the second set of dogbones, for example set 520, may be left at room-temperature in as-printed condition. Individual printing parameters may be varied systematically in 10% intervals ranging from −30% to +30% from an independently obtained set of nominally ideal values. These values may then be optimized for a-priori and found to produce near-full-density parts, greater than 99% dense, with acceptable mechanical properties and then may be used as the control group to demonstrate the LARS technique. In total, fifteen baseline control samples along with seventeen groups of five samples with variations in laser power, speed, and spot-size, relative to the control, are created and summarized in the tables as shown below.

TABLE 1
Each group of samples is created with a unique set
of printing parameters that results in slight variations
in their microstructure and porosity.

| | Parameter variation | | | | | | |
|---|---|---|---|---|---|---|---|
| | | | | 0% | | | |
| | −30% | −20% | −10% | (control) | +10% | +20% | +30 |
| Laser Power | 5 | 5 | 5 | 5 | 5 | 5 | |
| Laser Speed | 5 | 5 | 5 | 5 | 5 | 5 | 5 |
| Laser Spot size | 5 | 5 | 5 | 5 | 5 | 5 | 5 |

Although the dogbones 500 may have internal variances in their microstructure, the outward appearances of the dogbones are nearly identical. The average dimensions and standard deviations of the 200 dogbones are shown in Table 2 below. The dimensions and weights of the dogbones are close to each other and thus differences in the resonant frequency spectra and unlikely to originate from geometric or mass differences. As discussed herein, the LARS technique is useful for detecting internal differences in the printed objects/parts, such as the dogbones 500, 510, and 520, which appear identical to the naked eye.

TABLE 2
Average dimensions and standard deviations measured
from the 200 additively manufactured dogbones.

| | Grip Width | Gauge Width | Thickness | Length | Weight |
|---|---|---|---|---|---|
| | (mm) | (mm) | (mm) | (mm) | (g) |
| Average | 12.571 | 6.239 | 2.478 | 114.341 | 7.302 |
| Std. Dev. | 0.019 | 0.034 | 0.014 | 0.083 | 0.060 |

The variations in the printing parameters have a strong impact on the overall microstructure and defective content of the dogbones 500, ultimately affecting their mechanical properties. Via this experiment, it has been demonstrated that LARS technique can be used to non-destructively evaluate the defect content, namely porosity, in the dogbone samples 500 that result from changing printing parameters and guide the parameter optimization process for identifying ideal printing parameters for creating fully dense parts. The porosity in the dogbones can be further characterized using X-ray computed tomography (CT) scans with a resolution of 30 microns. Representative samples from each of the unique eighteen groups are scanned in the center of the gauge length along the shape 530 of the dogbone sample, shown in FIG. 5B. The CT scan may allow for the digitized 3D visualization and quantification of the total pore volume in the region of interest. It is expected that the scanned regions are representative of the entire dogbone volumes for each group, respectively.

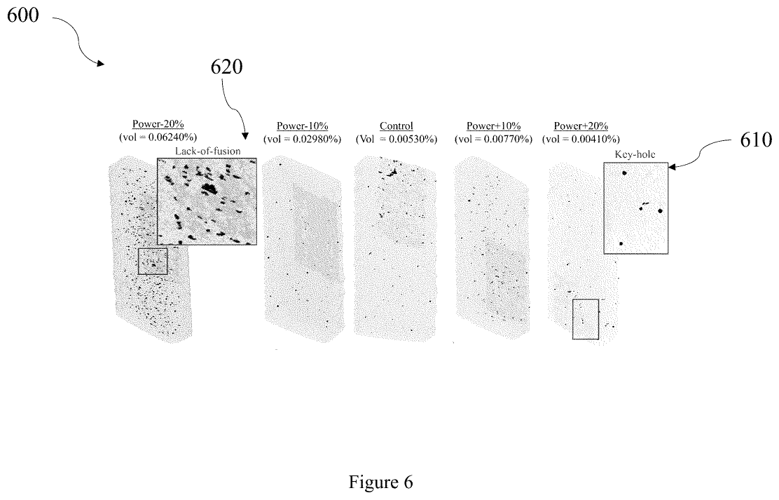

FIG. 6 depicts a plurality of X-ray computed tomography (CT) scans 600 showing porosity evolution in samples created with different relative laser power using laser powder bed fusion, in accordance with various embodiments. The CT scans 600 are performed in the center of each dogbone sample, in the middle of the gauge length and encompassing the entire gauge width. As such, it may be reasonable and expected that the microstructure and porosity are consistent throughout an entire dogbone. As shown in FIG. 6, the CT scans 600 demonstrate that the variations in porosity for the samples created with laser power variance of power−20%, power−10%, control, power+10%, and power+20%, respectively, as shown in FIG. 6. The control sample for the power variation group is observed to have a pore volume of 0.0053%, which is reasonably close to a fully dense material >99.9%. When the laser power is increased by 20%, relative to the laser power used to manufacture the control, the pore volume decreased to 0.0041%; however, large circular key-hole defects shown in an insert 610 can be observed, which are characteristic of parts additively manufactured with excessive laser power that creates unstable melt pools that eventually trap gases. Conversely, when the laser power was decreased by 20%, the pore volume increased to 0.0624%; lack-of-fusion defects, as shown in an insert 620, can be characterized by oblong shaped pores, are observed, and likely originated from the insufficient laser power to properly melt the powders.

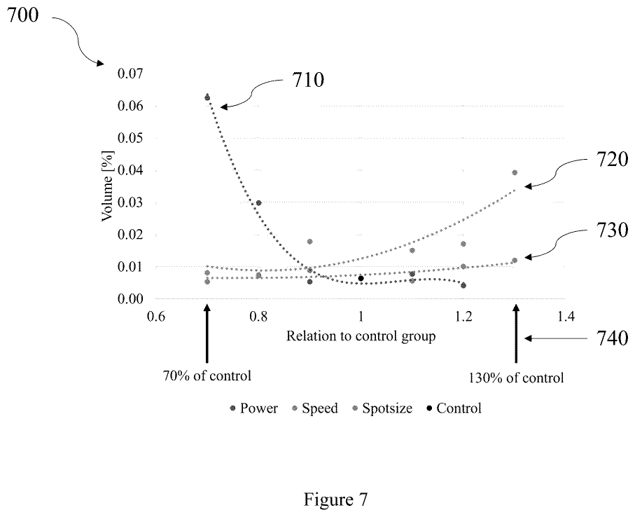

FIG. 7 shows a plot 700 depicting pore volume for all the groups of dogbone samples 500, which have been measured and plotted together to illustrate the porosity evolution in the samples as functions of the printing parameters, in accordance with various embodiments. As depicted in the plot 700, the pore volumes of the laser power variation group are shown in blue markers 710, the laser speed group in orange markers 720, the laser spotsize group in green markers 730, and the control group in black 740, respectively. Best fit curves have been added to highlight the trends. It is apparent, from the plot 700, that decreasing the laser power (blue curve with markers 710) results in greater pore volumes, while increases in the laser speed (orange curve with markers 720) leads to greater pore volumes. Evidently, changing the laser spotsize had minimal effect on the overall porosity, however, there is a small increase in porosity as the spotsize increases, relative to the spotsize used to manufacture the control sample.

As described with respect to FIG. 5, LARS technique can be used to obtain the resonant frequency spectra of all 200 dogbone samples 500. The spectra for the control groups can then be averaged together to get a baseline reference spectrum for heat treated set 510 and as-printed set 520, respectively. The SS of each dogbone can then be calculated with respect to the reference spectrum of their respective set, according to Eq. 4. The SS of the five samples in each group, seen in Table 1. The number of samples in each 100-sample set created and their variations in printing parameters relative to the control. As discussed earlier, in total, two sets of 100 samples are created, one to be heat treated and the other to be left in the as-printed condition, which are then averaged together to obtain an average SS for the unique set of printing parameters. The average SS for the heat treated and as-printed groups are calculated separately and plotted together.

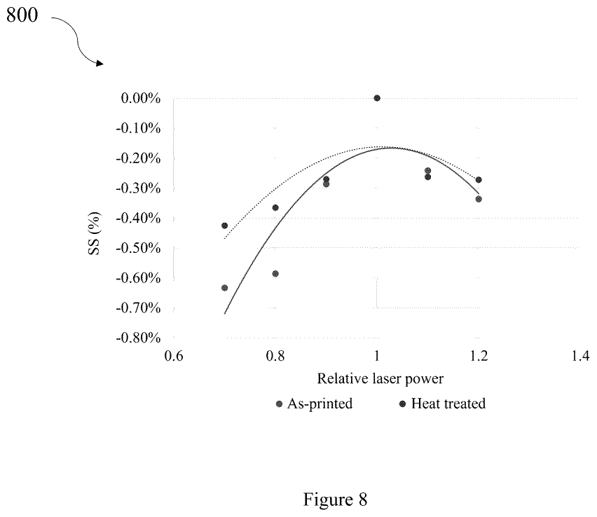

FIG. 8 shows a plot 800 depicting for the average SS for the heat treated and as-printed groups of dogbone samples 500 based on laser power variation relative to the laser power used to manufacture the control dogbones, in accordance with various embodiments. In FIG. 8, it can be observed from the plot 800 that the peak positions of parts made with lower laser power become increasingly more downshifted relative to the peak positions of the control group dogbones, as evident by the average SS values. This correlates strongly with the trend of increasing porosity observed in the parts due to the lower laser power, as illustrated by the blue curves in FIG. 7.

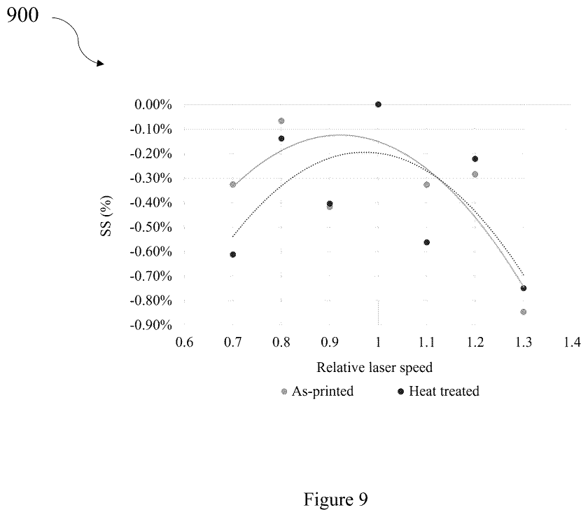

FIG. 9 shows a plot 900 depicting for the average SS for the heat treated and as-printed groups of dogbone samples 500 based on laser speed variation relative to the laser speed used to manufacture the control dogbones, in accordance with various embodiments. Similarly, the trends observed in the plot 900 of FIG. 9 show that larger LARS measured peak downshifts with increasing laser speed. Again, the average SS correlate strongly with the increasing porosity due to increasing laser speeds, shown by the orange curves in FIG. 7.

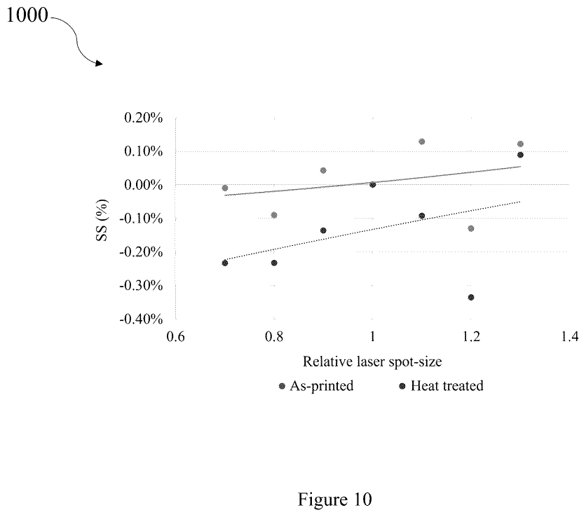

FIG. 10 shows a plot 1000 depicting for the average SS for the heat treated and as-printed groups of dogbone samples 500 based on laser spotsize variation relative to the laser spotsize used to manufacture the control dogbones, in accordance with various embodiments. in particular, the plot 1000 of FIG. 10 illustrates that the LARS measured SS generally increases with increasing laser spotsize. However, the magnitude of the SS is much smaller than the SS's observed in the laser power and speed groups, indicating that laser spotsize has a smaller effect on the porosity than the other two printing parameters. This also correlates strongly with the trends observed for the porosity evolution in the dogbones as spotsize was increased, seen by the green curve in FIG. 7.

Overall, the strong correlation in trends between the LARS measured average SS in the 200 dogbones indicate that LARS measurements can be directly used to evaluate the porosity in parts made by laser powder bed fusion in all three groups, laser power, laser speed, and laser spotsize. The set of printing parameters that resulted in the least amount of SS can be observed to also have the least porosity, demonstrating how LARS can be used to identify the optimal set of printing parameters to achieve the densest parts. Notably, the resolution of the LARS technique is high, which allows it to detect minute porosity differences in parts that are greater than 99.9% dense. Furthermore, parts with larger SS can be expected to have more porosity, which may be undesirable in some applications. It should also be noted that LARS can consistently detect differences between identical parts, printed with identical parameters, but having different heat treatment histories, as indicated by the red curves in FIG. 8, FIG. 9, and FIG. 10.

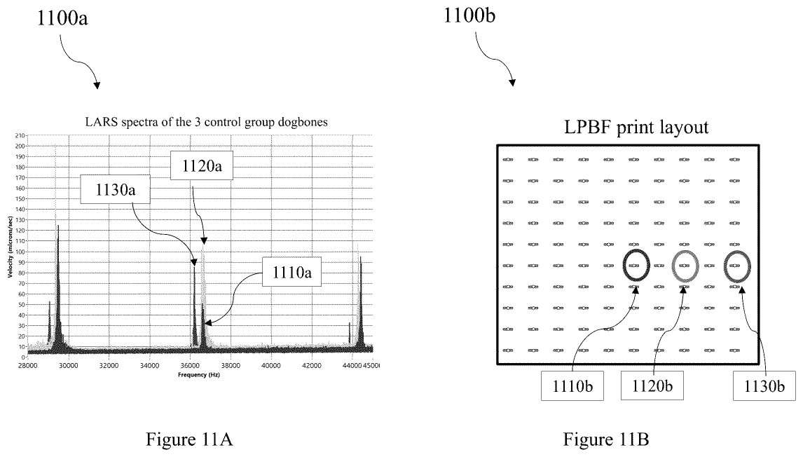

FIG. 11A shows a plot 1100a depicting partial LARS measured resonant frequency spectra 1110a, 1120a, and 1130a, respectively, of three control group dogbones 1110b, 1120b, and 1130b, overlayed together, in accordance with various embodiments. FIG. 11B shows a laser powder bed fusion (LPBF) print layout 1100b of the printed dogbones, in accordance with various embodiments. The LPBF print layout 1100b is shown with the edges of the print bed outlined in black and the locations of the three dogbones 1110b, 1120b, and 1130b are circled in red, green and blue, corresponding to the colored curves 1110a, 1120a, and 1130a shown on the plot 1100a.

The plot 1100a of FIG. 11A further depicts the LARS spectra of three control group samples from 28-45 kHz. The three spectra have been chosen from dogbones that are identical in shape, weight, and printing parameters, but differ in their location on the print bed. The red curve 1110a represents the center most dogbone, while the blue curve 1130a represents the outermost dogbone, and the green curve 1120a lies in the middle between the center and the outermost dogbone. A clear gradual distinction between their resonant frequency spectra can be observed, with the outermost dogbone 1130b (in blue) having peak positions that are more downshifted relative to the centermost dogbone 1110b (in red). This demonstrates that LARS can be used to identify dogbones that arise from different locations on the 3D print bed, in accordance with various embodiments.

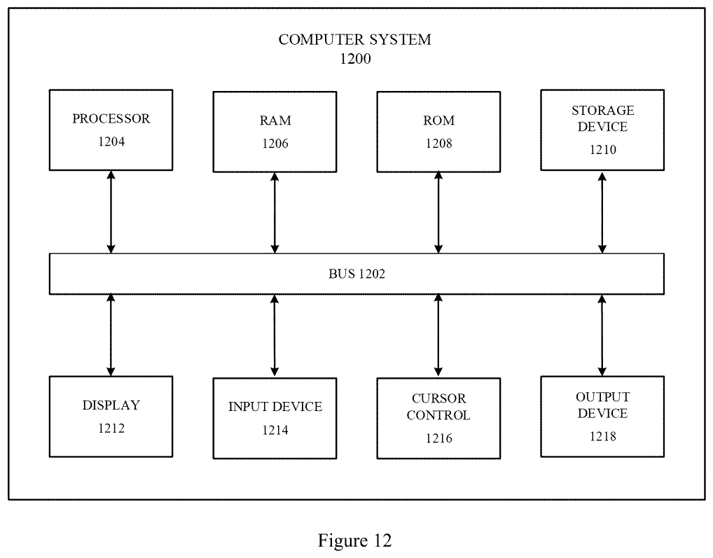

FIG. 12 is a block diagram illustrating an example computer system 1200, with which embodiments of the disclosed system and method for performing non-destructive investigations may be implemented, in accordance with various embodiments. For example, the illustrated computer system 1200 can be a local or remote computer system operatively connected to the disclosed system and method for performing non-destructive investigations of an object, such as those described with respect to FIGS. 1-11, 13, and 14.

In various embodiments of the present teachings, computer system 1200 can include a bus 1202 or other communication mechanism for communicating information and a processor 1204 coupled with bus 1202 for processing information. In various embodiments, computer system 1200 can also include a memory, which can be a random-access memory (RAM) 1206 or other dynamic storage device, coupled to bus 1202 for determining instructions to be executed by processor 1204. Memory can also be used for storing temporary variables or other intermediate information during execution of instructions to be executed by processor 1204. In various embodiments, computer system 1200 can further include a read only memory (ROM) 1208 or other static storage device coupled to bus 1202 for storing static information and instructions for processor 1204. A storage device 1210, such as a magnetic disk or optical disk, can be provided and coupled to bus 1202 for storing information and instructions.

In various embodiments, computer system 1200 can be coupled via bus 1202 to a display 1212, such as a cathode ray tube (CRT) or liquid crystal display (LCD), for displaying information to a computer user. An input device 1214, including alphanumeric and other keys, can be coupled to bus 1202 for communication of information and command selections to processor 1204. Another type of user input device is a cursor control 1216, such as a mouse, a trackball or cursor direction keys for communicating direction information and command selections to processor 1204 and for controlling cursor movement on display 1212. This input device 1214 typically has two degrees of freedom in two axes, a first axis (i.e., x) and a second axis (i.e., y), that allows the device to specify positions in a plane. However, it should be understood that input devices 1214 allowing for 3-dimensional (x, y and z) cursor movement are also contemplated herein. In accordance with various embodiments, components 1212/1214/1216, together or individually, can make up a control system that connects the remaining components of the computer system to the systems herein and methods conducted on such systems, and controls execution of the methods and operation of the associated system.

In various embodiments, the computer system 1200 includes an output device 1218. In various embodiments, the output device 1218 can be a wireless device, a computing device, a portable computing device, a communication device, a printer, a graphical user interface (GUI), a gaming controller, a joy-stick controller, an external display, a monitor, a mixed reality device, an artificial reality device, or a virtual reality device.

Consistent with certain implementations of the present teachings, results can be provided by computer system 1200 in response to processor 1204 executing one or more sequences of one or more instructions contained in memory 1206. Such instructions can be read into memory 1206 from another computer-readable medium or computer-readable storage medium, such as storage device 1210. Execution of the sequences of instructions contained in memory 1206 can cause processor 1204 to perform the processes described herein. Alternatively, hard-wired circuitry can be used in place of or in combination with software instructions to implement the present teachings. Thus, implementations of the present teachings are not limited to any specific combination of hardware circuitry and software.

The term “computer-readable medium” (e.g., data store, data storage, etc.) or “computer-readable storage medium” as used herein refers to any media that participates in providing instructions to processor 1204 for execution. Such a medium can take many forms, including but not limited to, non-volatile media, volatile media, and transmission media. Examples of non-volatile media can include, but are not limited to, dynamic memory, such as memory 1206. Examples of transmission media can include, but are not limited to, coaxial cables, copper wire, and fiber optics, including the wires that comprise bus 1202.

Common forms of computer-readable media include, for example, a floppy disk, a flexible disk, hard disk, magnetic tape, or any other magnetic medium, a CD-ROM, any other optical medium, punch cards, paper tape, any other physical medium with patterns of holes, a RAM, PROM, and EPROM, a FLASH-EPROM, another memory chip or cartridge, or any other tangible medium from which a computer can read.

In addition to computer-readable medium, instructions or data can be provided as signals on transmission media included in a communications apparatus or system to provide sequences of one or more instructions to processor 1204 of computer system 1200 for execution. For example, a communication apparatus may include a transceiver having signals indicative of instructions and data. The instructions and data are configured to cause one or more processors to implement the functions outlined in the disclosure herein. Representative examples of data communications transmission connections can include, but are not limited to, telephone modem connections, wide area networks (WAN), local area networks (LAN), infrared data connections, NFC connections, etc.

It should be appreciated that the methodologies described herein, flow charts, diagrams and accompanying disclosure can be implemented using computer system 1200 as a standalone device or on a distributed network or shared computer processing resources such as a cloud computing network.

The methodologies described herein may be implemented by various means depending upon the application. For example, these methodologies may be implemented in hardware, firmware, software, or any combination thereof. For a hardware implementation, the processing unit may be implemented within one or more application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, electronic devices, other electronic units designed to perform the functions described herein, or a combination thereof.

In various embodiments, the methods of the present teachings may be implemented as firmware and/or a software program and applications written in conventional programming languages such as C, C++, Python, etc. If implemented as firmware and/or software, the embodiments described herein can be implemented on a non-transitory computer-readable medium in which a program is stored for causing a computer to perform the methods described above. It should be understood that the various engines described herein can be provided on a computer system, such as computer system 1200, whereby processor 1204 would execute the analyses and determinations provided by these engines, subject to instructions provided by any one of, or a combination of, memory components 1206/1208/1210 and user input provided via input device 1214.

While the present teachings are described in conjunction with various embodiments, it is not intended that the present teachings be limited to such embodiments. On the contrary, the present teachings encompass various alternatives, modifications, and equivalents, as will be appreciated by those of skill in the art. In describing the various embodiments, the specification may have presented a method and/or process as a particular sequence of steps. However, to the extent that the method or process does not rely on the particular order of steps set forth herein, the method or process should not be limited to the particular sequence of steps described, and one skilled in the art can readily appreciate that the sequences may be varied and still remain within the spirit and scope of the various embodiments.

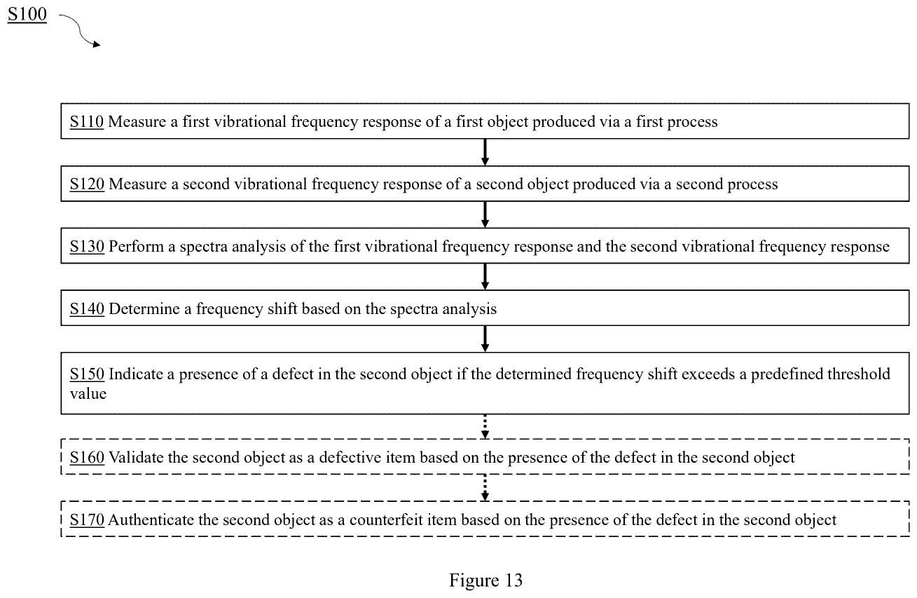

FIG. 13 illustrates a flowchart for a method S100, in accordance with one or more embodiments. In one or more embodiments, the method S100 may be used for non-destructive characterizations of objects using diagnostic techniques as disclosed herein. As illustrated in FIG. 13, the method S100 includes, at step S110, measuring a first vibrational frequency response of a first object produced via a first process; at step S120, measuring a second vibrational frequency response of a second object produced via a second process; at step S130, performing a spectra analysis of the first vibrational frequency response and the second vibrational frequency response; at step S140, determining a frequency shift based on the spectra analysis; and at step S150, indicating a presence of a defect in the second object if the determined frequency shift exceeds a predefined threshold value.

In various embodiments of the method S100, performing the spectra analysis of the first vibrational frequency response and the second vibrational frequency response may include identifying a first plurality of peaks from the first vibrational frequency response as resonant frequency modes of the first object, identifying a second plurality of peaks from the second vibrational frequency response as resonant frequency modes of the second object, for each resonant frequency mode of the first (second) object, comparing a peak position of a corresponding peak of the first plurality and a peak position of a corresponding peak of the second plurality, for each resonant frequency mode of the first (second) object, determining a frequency difference between the two peak positions, and averaging all determined frequency differences to provide the frequency shift for the first vibrational frequency response and the second vibrational frequency response.

In one or more embodiments of the method S100, the first object and the second object may have a substantially similar geometrical shape or a substantially similar volume. In one or more embodiments, the first object and the second object may have a substantially similar geometrical shape, a substantially similar volume, and a substantially similar material composition.

In one or more embodiments of the method S100, the first object and the second object may have the same metals. In one or more embodiments, the first object and the second object may have different heat treatment profiles or processing history. In one or more embodiments, the defect in the second object may include a void, a crack, or a plurality of pores. In one or more embodiments, the presence of the defect in the second object may indicate that the defect is a physical defect in the second object and that the physical defect is not present in the first object.

In one or more embodiments of the method S100, the first process is a metallurgical process, and the second process is an additive manufacturing process. In one or more embodiments, the first process and the second process are both metallurgical processes. In one or more embodiments, the first process is a first additive manufacturing process using a laser-based three-dimensional (3D) printer with a first set of manufacturing parameters, the second process is a second additive manufacturing process using the laser-based 3D printer with a second set of manufacturing parameters, and the first set of manufacturing parameters may differ from the second set of manufacturing parameters in laser power, laser raster speed, laser spot size, or a combination thereof.

In one or more embodiments of the method S100, the presence of the defect in the second object may indicate a difference in porosity between the first object and the second object.

As illustrated in FIG. 13, the method S100 may optionally include, at step S160, validating the second object as a defective item based on the presence of the defect in the second object.

Furthermore, the method S100 may optionally include, at step S170, authenticating the second object as a counterfeit item based on the presence of the defect in the second object.

In one or more embodiments of the method S100, the first vibrational frequency response of the first object and the second vibrational frequency response of the second object are measured via a laser acoustic resonance spectroscopy system using a laser doppler vibrometer. In one or more embodiments, the first vibrational frequency response of the first object and the second vibrational frequency response of the second object are measured in an acoustic frequency range between 1 Hz and 60 kHz.

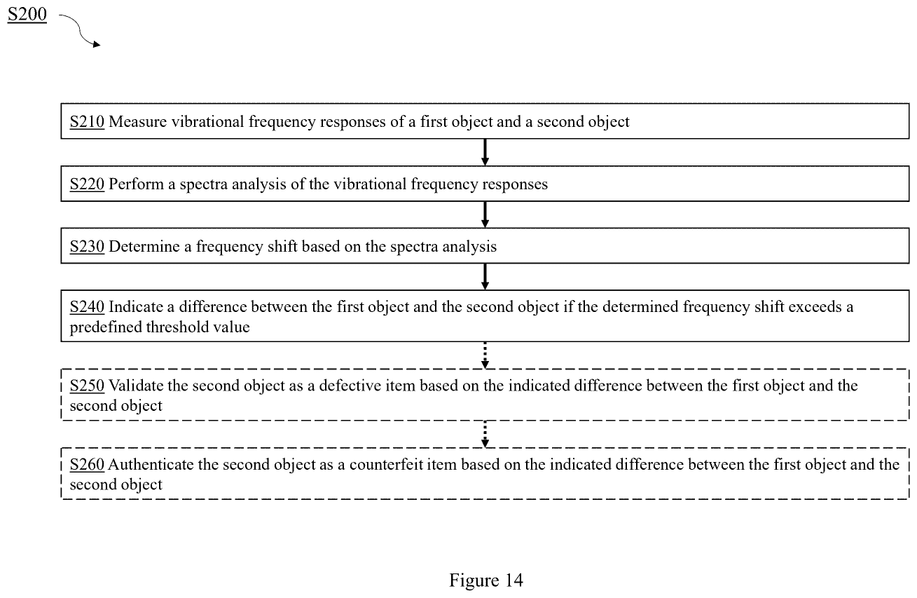

FIG. 14 illustrates a flowchart for another method S200, in accordance with one or more embodiments. In one or more embodiments, the method S200 may be used for non-destructive characterizations of objects using diagnostic techniques as disclosed herein. As illustrated in FIG. 14, the method S200 includes, at step S210, measuring vibrational frequency responses of a first object and a second object; at step S220, performing a spectra analysis of the vibrational frequency responses; at step S230, determining a frequency shift based on the spectra analysis; and at step S240, indicating a difference between the first object and the second object if the determined frequency shift exceeds a predefined threshold value.

In various embodiments of the method S200, performing the spectra analysis of the vibrational frequency responses may further include identifying a plurality of peaks from the vibrational frequency responses; pairing corresponding peaks originating from the first object and the second object; designating the paired corresponding peaks as resonant frequency modes of the first object and second object; for each resonant frequency mode of the first object and the second object, determining a frequency difference for each of the paired corresponding peaks; and averaging all determined frequency differences to provide the frequency shift.

In one or more embodiments of the method S200, the first object and the second object may have a substantially similar geometrical shape or a substantially similar volume. In one or more embodiments, the first object and the second object may have a substantially similar geometrical shape, a substantially similar volume, and a substantially similar material composition. In one or more embodiments, the first object and the second object may have the same metals. In one or more embodiments, the first object and the second object may have different heat treatment profiles or processing history.

In one or more embodiments of the method S200, the difference between the first object and the second object may indicate a presence of a void, a crack, or a plurality of pores in the second object. In one or more embodiments, the difference between the first object and the second object may indicate a physical defect in the second object and that the physical defect is not present in the first object.

In one or more embodiments of the method S200, the first object may be produced via a metallurgical process, and the second object may be produced via an additive manufacturing process. In one or more embodiments, the first object may be produced via a laser-based three-dimensional (3D) printer with a first set of manufacturing parameters, the second object may be produced via the laser-based 3D printer with a second set of manufacturing parameters, and the first set of manufacturing parameters may differ from the second set of manufacturing parameters in laser power, laser raster speed, laser spot size, or a combination thereof.

In one or more embodiments of the method S200, the difference between the first object and the second object may indicate a difference in porosity between the first object and the second object.

As illustrated in FIG. 14, the method S200 may optionally include, at step S250, validating the second object as a defective item based on the indicated difference between the first object and the second object.

Furthermore, the method S200 may optionally include, at step S260, authenticating the second object as a counterfeit item based on the indicated difference between the first object and the second object.

In one or more embodiments of the method S200, the vibrational frequency responses of the first object and the second object may be measured via a laser acoustic resonance spectroscopy system using a laser doppler vibrometer. In one or more embodiments, the vibrational frequency responses of the first object and the second object may be measured in an acoustic frequency range between 1 Hz and 60 kHz.

### EMBODIMENTS

Embodiment 1. A method comprising: measuring a first vibrational frequency response of a first object produced via a first process; measuring a second vibrational frequency response of a second object produced via a second process; performing a spectra analysis of the first vibrational frequency response and the second vibrational frequency response; determining a frequency shift based on the spectra analysis; and indicating a presence of a defect in the second object if the determined frequency shift exceeds a predefined threshold value.
Embodiment 2. The method of Embodiment 1, wherein performing the spectra analysis of the first vibrational frequency response and the second vibrational frequency response comprises: identifying a first plurality of peaks from the first vibrational frequency response as resonant frequency modes of the first object; identifying a second plurality of peaks from the second vibrational frequency response as resonant frequency modes of the second object; for each resonant frequency mode of the first (second) object, comparing a peak position of a corresponding peak of the first plurality and a peak position of a corresponding peak of the second plurality; for each resonant frequency mode of the first (second) object, determining a frequency difference between the two peak positions; and averaging all determined frequency differences to provide the frequency shift for the first vibrational frequency response and the second vibrational frequency response.
Embodiment 3. The method of Embodiments 1 or 2, wherein the first object and the second object have a substantially similar geometrical shape or a substantially similar volume.
Embodiment 4. The method of Embodiments 1 or 2, wherein the first object and the second object have a substantially similar geometrical shape, a substantially similar volume, and a substantially similar material composition.
Embodiment 5. The method of any one of Embodiments 1-4, wherein the first object and the second object have same metals.
Embodiment 6. The method of any one of Embodiments 1-5, wherein the first object and the second object have different heat treatment profiles or processing history.
Embodiment 7. The method of any one of Embodiments 1-6, wherein the defect in the second object comprises a void, a crack, or a plurality of pores.
Embodiment 8. The method of any one of Embodiments 1-7, wherein the presence of the defect in the second object indicates that the defect is a physical defect in the second object and that the physical defect is not present in the first object.
Embodiment 9. The method of any one of Embodiments 1-8, wherein the first process is a metallurgical process, and the second process is an additive manufacturing process.
Embodiment 9-1. The method of any one of Embodiments 1-8, wherein the first process and the second process are both metallurgical processes.
Embodiment 10. The method of any one of Embodiments 1-8, wherein: the first process is a first additive manufacturing process using a laser-based three-dimensional (3D) printer with a first set of manufacturing parameters, the second process is a second additive manufacturing process using the laser-based 3D printer with a second set of manufacturing parameters, and the first set of manufacturing parameters differ from the second set of manufacturing parameters in laser power, laser raster speed, laser spot size, or a combination thereof.
Embodiment 11. The method of any one of Embodiments 1-10, wherein the presence of the defect in the second object indicates a difference in porosity or material composition between the first object and the second object.
Embodiment 12. The method of any one of Embodiments 1-11, further comprising:
   validating the second object as a defective item based on the presence of the defect in the second object.
Embodiment 13. The method of any one of Embodiments 1-12, further comprising: authenticating the second object as a counterfeit item based on the presence of the defect in the second object.
Embodiment 14. The method of any one of Embodiments 1-13, wherein the first vibrational frequency response of the first object and the second vibrational frequency response of the second object are measured via a laser acoustic resonance spectroscopy system using a laser doppler vibrometer.
Embodiment 15. The method of any one of Embodiments 1-14, wherein the first vibrational frequency response of the first object and the second vibrational frequency response of the second object are measured in an acoustic frequency range between 1 Hz and 60 KHz.
Embodiment 16. A system comprising: a laser doppler vibrometer configured to measure a vibrational response of a first object and a second object; a processor and a non-transitory computer readable medium operably coupled thereto, the processor operationally coupled and configured to control the laser doppler vibrometer and to acquire data from the laser doppler vibrometer, wherein the non-transitory computer readable medium comprising a plurality of instructions stored in association therewith that are accessible to, and executable by, the processor, to perform one or more operations, which comprise: acquiring a first vibrational frequency response of the first object; acquiring a second vibrational frequency response of the second object; performing a spectra analysis of the first vibrational frequency response and the second vibrational frequency response; determining a frequency shift based on the spectra analysis; and indicating a presence of a defect in the second object if the determined frequency shift exceeds a predefined threshold value.
Embodiment 17. The system of Embodiment 16, wherein performing the spectra analysis of the first vibrational frequency response and the second vibrational frequency response comprises: identifying a first plurality of peaks from the first vibrational frequency response as resonant frequency modes of the first object; identifying a second plurality of peaks from the second vibrational frequency response as resonant frequency modes of the second object; for each resonant frequency mode of the first (second) object, comparing a peak position of a corresponding peak of the first plurality and a peak position of a corresponding peak of the second plurality; for each resonant frequency mode of the first (second) object, determining a frequency difference between the two peak positions; and averaging all determined frequency differences to provide the frequency shift for the first vibrational frequency response and the second vibrational frequency response.
Embodiment 18. The system of Embodiments 16 or 17, further comprising: an elastic mesh net configured to mount the first object or the second object; and a piezoelectric transducer disposed underneath the elastic mesh net and configured to vibrate the first object or the second object during a vibrational response measurement performed by the laser doppler vibrometer.
Embodiment 19. The system of any one of Embodiments 16-18, wherein the presence of the defect in the second object indicates: a void, a crack, or a plurality of pores, a difference in porosity or material composition between the first object and the second object, that the defect is a physical defect in the second object and that the physical defect is not present in the first object, or a combination thereof.
Embodiment 20. The system of any one of Embodiments 16-19, wherein the presence of the defect in the second object indicates that the second object is a defective item or a counterfeit item.
Embodiment 21. The system of any one of Embodiments 16-20, wherein the laser doppler vibrometer is further configured to measure the vibrational response in an acoustic frequency range between 1 Hz and 60 kHz.
Embodiment 22. A method comprising: measuring vibrational frequency responses of a first object and a second object; performing a spectra analysis of the vibrational frequency responses; determining a frequency shift based on the spectra analysis; and indicating a difference between the first object and the second object if the determined frequency shift exceeds a predefined threshold value.
Embodiment 23. The method of Embodiment 22, wherein performing the spectra analysis of the vibrational frequency responses comprises: identifying a plurality of peaks from the vibrational frequency responses; pairing corresponding peaks originating from the first object and the second object; designating the paired corresponding peaks as resonant frequency modes of the first object and second object; for each resonant frequency mode of the first object and the second object, determining a frequency difference for each of the paired corresponding peaks; and averaging all determined frequency differences to provide the frequency shift.
Embodiment 24. The method of Embodiments 22 or 23, wherein the first object and the second object have a substantially similar geometrical shape or a substantially similar volume.
Embodiment 25. The method of Embodiments 22 or 23, wherein the first object and the second object have a substantially similar geometrical shape, a substantially similar volume, and a substantially similar material composition.
Embodiment 26. The method of any one of Embodiments 22-25, wherein the first object and the second object have same metals.
Embodiment 27. The method of any one of Embodiments 22-26, wherein the first object and the second object have different heat treatment profiles or processing history.
Embodiment 28. The method of any one of Embodiments 22-27, wherein the difference between the first object and the second object indicates a presence of a void, a crack, or a plurality of pores in the second object.
Embodiment 29. The method of any one of Embodiments 22-28, wherein the difference between the first object and the second object indicates a physical defect in the second object and that the physical defect is not present in the first object.
Embodiment 30. The method of any one of Embodiments 22-29, wherein the first object is produced via a metallurgical process, and the second object is produced via an additive manufacturing process.
Embodiment 31. The method of any one of Embodiments 22-29, wherein: the first object is produced via a laser-based three-dimensional (3D) printer with a first set of manufacturing parameters, the second object is produced via the laser-based 3D printer with a second set of manufacturing parameters, and the first set of manufacturing parameters differ from the second set of manufacturing parameters in laser power, laser raster speed, laser spot size, or a combination thereof.
Embodiment 32. The method of any one of Embodiments 22-31, wherein the difference between the first object and the second object indicates a difference in porosity between the first object and the second object.
Embodiment 33. The method of any one of Embodiments 22-32, further comprising: validating the second object as a defective item based on the indicated difference between the first object and the second object.
Embodiment 34. The method of any one of Embodiments 22-33, further comprising: authenticating the second object as a counterfeit item based on the indicated difference between the first object and the second object.
Embodiment 35. The method of any one of Embodiments 22-34, wherein the vibrational frequency responses of the first object and the second object are measured via a laser acoustic resonance spectroscopy system using a laser doppler vibrometer.
Embodiment 36. The method of any one of Embodiments 22-35, wherein the vibrational frequency responses of the first object and the second object are measured in an acoustic frequency range between 1 Hz and 60 kHz.

What is claimed is:

1. A method comprising:
measuring a first vibrational frequency response of a first object produced via a first process;
measuring a second vibrational frequency response of a second object produced via a second process;
performing a spectra analysis of the first vibrational frequency response and the second vibrational frequency response;
determining a frequency shift based on the spectra analysis; and
indicating a presence of a defect in the second object if the determined frequency shift exceeds a predefined threshold value.

2. The method of claim 1, wherein performing the spectra analysis of the first vibrational frequency response and the second vibrational frequency response comprises:
identifying a first plurality of peaks from the first vibrational frequency response as resonant frequency modes of the first object;
identifying a second plurality of peaks from the second vibrational frequency response as resonant frequency modes of the second object;
for each resonant frequency mode of the first (second) object, comparing a peak position of a corresponding peak of the first plurality and a peak position of a corresponding peak of the second plurality;
for each resonant frequency mode of the first (second) object, determining a frequency difference between the two peak positions; and
averaging all determined frequency differences to provide the frequency shift for the first vibrational frequency response and the second vibrational frequency response.

3. The method of claim 1, wherein the first object and the second object have a substantially similar geometrical shape, a substantially similar volume, and/or a substantially similar material composition.

4. The method of claim 1, wherein the first object and the second object have different heat treatment profiles or processing history.

5. The method of claim 1, wherein the defect in the second object comprises a void, a crack, or a plurality of pores.

6. The method of claim 1, wherein the presence of the defect in the second object indicates that the defect is a physical defect in the second object and that the physical defect is not present in the first object.

7. The method of claim 1, wherein the first process is a metallurgical process, and the second process is an additive manufacturing process.

8. The method of claim 1, wherein:
the first process is a first additive manufacturing process using a laser-based three-dimensional (3D) printer with a first set of manufacturing parameters,
the second process is a second additive manufacturing process using the laser-based 3D printer with a second set of manufacturing parameters, and
the first set of manufacturing parameters differ from the second set of manufacturing parameters in laser power, laser raster speed, laser spot size, or a combination thereof.

9. The method of claim 1, wherein the presence of the defect in the second object indicates a difference in porosity between the first object and the second object.

10. The method of claim 1, further comprising:
validating the second object as a defective item based on the presence of the defect in the second object, and/or
authenticating the second object as a counterfeit item based on the presence of the defect in the second object.

11. A system comprising:
a laser doppler vibrometer configured to measure a vibrational response of a first object and a second object;
a processor and a non-transitory computer readable medium operably coupled thereto, the processor operationally coupled and configured to control the laser doppler vibrometer and to acquire data from the laser doppler vibrometer, wherein the non-transitory computer readable medium comprising a plurality of instructions stored in association therewith that are accessible to, and executable by, the processor, to perform one or more operations, which comprise:
acquiring a first vibrational frequency response of the first object;
acquiring a second vibrational frequency response of the second object;
performing a spectra analysis of the first vibrational frequency response and the second vibrational frequency response;
determining a frequency shift based on the spectra analysis; and
indicating a presence of a defect in the second object if the determined frequency shift exceeds a predefined threshold value.

12. The system of claim 11, wherein performing the spectra analysis of the first vibrational frequency response and the second vibrational frequency response comprises:
identifying a first plurality of peaks from the first vibrational frequency response as resonant frequency modes of the first object;
identifying a second plurality of peaks from the second vibrational frequency response as resonant frequency modes of the second object;
for each resonant frequency mode of the first (second) object, comparing a peak position of a corresponding peak of the first plurality and a peak position of a corresponding peak of the second plurality;
for each resonant frequency mode of the first (second) object, determining a frequency difference between the two peak positions; and
averaging all determined frequency differences to provide the frequency shift for the first vibrational frequency response and the second vibrational frequency response.

13. The system of claim 11, further comprising:
an elastic mesh net configured to mount the first object or the second object; and
a piezoelectric transducer disposed underneath the elastic mesh net and configured to vibrate the first object or the second object during a vibrational response measurement performed by the laser doppler vibrometer.

14. The system of claim 11, wherein the presence of the defect in the second object indicates:
a void, a crack, or a plurality of pores,
a difference in porosity or material composition between the first object and the second object,
that the defect is a physical defect in the second object and that the physical defect is not present in the first object, or
a combination thereof.

15. The system of claim 11, wherein the presence of the defect in the second object indicates that the second object is a defective item or a counterfeit item.

16. A method comprising:
measuring vibrational frequency responses of a first object and a second object;
performing a spectra analysis of the vibrational frequency responses;
determining a frequency shift based on the spectra analysis; and
indicating a difference between the first object and the second object if the determined frequency shift exceeds a predefined threshold value.

17. The method of claim 16, wherein performing the spectra analysis of the vibrational frequency responses comprises:
identifying a plurality of peaks from the vibrational frequency responses;
pairing corresponding peaks originating from the first object and the second object;
designating the paired corresponding peaks as resonant frequency modes of the first object and second object;
for each resonant frequency mode of the first object and the second object, determining a frequency difference for each of the paired corresponding peaks; and
averaging all determined frequency differences to provide the frequency shift.

18. The method of claim 16, wherein the difference between the first object and the second object indicates a presence of a void, a crack, or a plurality of pores in the second object.

19. The method of claim 16, wherein the difference between the first object and the second object indicates a physical defect in the second object and that the physical defect is not present in the first object.

20. The method of claim 16, further comprising:
validating the second object as a defective item based on the indicated difference between the first object and the second object, and/or
authenticating the second object as a counterfeit item based on the indicated difference between the first object and the second object.
